(19) United States
(12) Patent Application Publication
(10) Pub. No.: US 2025/0339591 A1
(43) Pub. Date: Nov. 6, 2025

(54) Leakproof Supplemental Nursing System

(71) Applicant: Victoria Flores, cypress, TX (US)

(72) Inventor: Victoria Flores, cypress, TX (US)

(21) Appl. No.: 18/655,223

(22) Filed: May 3, 2024

(51) Int. Cl. A61M 1/06; A61J 13/00

(52) U.S. Cl. CPC A61M 1/068; A61J 13/00

(57) ABSTRACT

Disclosed in the present invention is a leakproof supplemental nursing system, comprising a silicone squeeze pouch with an airtight seal and connecting a silicone tube with a flow controller. The system further comprises a leakproof BPA-free lid instead of the artificial nipple that is normally used including an attached airtight tube inserted that can be changed out every 24-48 hours for safety. It also has a silicone BPA-free milk storage pouch that is used for easy suction and cleaning. This pouch includes a handle for hanging on taller surfaces for easy gravitation. It has a thin tube that is easy to clean and has a flow control attachment that can adjust the supplemental milk from a drip flow to a medium or a faster flow.

### TECHNICAL FIELD

The present invention relates to a Supplemental nursing system, and in particular, to a leakproof Supplemental nursing system, comprising a silicone squeeze pouch with an air-tight seal and a connecting silicone tube with a flow controller.

### BACKGROUND ART

A Supplemental nursing system is a device that can assist mothers who are going through challenges with breastfeeding. The supplemental nursing system includes a small milk container, a thin tube, and an artificial nipple. The milk container is worn on the mother's neck, half of the end of the thin tube is taped to the mother's areola and the last end of the tube is inserted into the baby's mouth. When the baby feeds on the mother's nipple, the milk flows from the container through the tube and into the baby's mouth.

Supplemental nursing systems' aren't widely used by prospective breastfeeding mothers due to needing to pay a lactation consultant to train mothers on how to use the supplemental nursing system due to the steep learning curve. Due to all current supplemental nursing system products only using plastic, mothers need to replace the whole system daily. Another disadvantage is the waste and financial cost of formula leakage from the system.

Various systems and methods for the pressure-regulated transfer of medical fluids are disclosed. The US patent application published as US20230075991A1 in March 2023 discloses pressure-regulating fluid transfer systems and methods. The system includes an adapter assembly that connects with a medical container and a syringe assembly. The syringe assembly can include a first reservoir and a second reservoir. In various embodiments, when the adapter assembly and the syringe assembly are coupled, the first reservoir and the container can exchange regulating fluid and the second reservoir, and the container can exchange medical liquid from the medical container. However, it lacks a leakproof Supplemental nursing system as taught in the present invention.

The problem that a Supplemental nursing system solves is the apprehension of breastfeeding for families. For example, some mothers may have a milk production delay due to a post-partum hemorrhage, previous breast surgery, having a c-section, and more medical reasons. A Supplemental nursing system can help overcome these obstacles by providing supplemental milk to the baby while the baby learns how to latch. Here are some of the specific problems that a Leak-proof supplemental nursing system can solve:

Low milk supply: A Leak-proof supplemental nursing system can help to increase a mother's milk supply by providing supplemental milk to the baby without frustration. This can be especially helpful for mothers who are struggling to breastfeed or are returning to work soon.

Baby not latching: A Leak-proof supplemental nursing system can help a baby who is not latching effectively to get the milk they need temporarily. The supplemental milk can be delivered through a tube that is placed alongside the mother's nipple, so the baby can still suckle on the breast and receive the benefits of skin-to-skin contact.

Baby not sucking effectively: A Leak-proof supplemental nursing system can also help a baby who is not sucking effectively to get the milk they need. The supplemental milk can be delivered through a tube that is placed in the baby's mouth, so the baby can still get the milk they need without having to suck as hard. Therefore, the mother's nipple won't bleed or become sore.

Promote bonding: Breastfeeding is a great way to connect with your baby, and a Leakproof supplemental nursing system can help to make this once in their lifetime experience even more special. The baby can still suck on the breast and get the benefits of skin-to-skin bonding, even if they are also getting supplemental milk.

Easy to use: The Leakproof supplemental nursing system is an easy set-up, taking less than a minute each time. There's no learning curve, so no need for a lactation consultant to set up.

Increase milk production: The act of breastfeeding can help to increase milk production and a Leak-proof supplemental nursing system can help to make this process even more effective. The supplemental milk can help to stimulate the mother's breasts, which can lead to an increase in milk production.

Help babies gain weight: Babies who are not getting enough milk may not gain weight and are in a low percentile for growth. A Leak-proof supplemental nursing system can help to ensure that these babies are getting the milk they need, which can help them gain weight and grow healthy.

Save money: A Leak proof supplemental nursing system can help lower costs by not having to purchase as much formula due to no leakage, with the Leak-proof supplemental nursing system, you purchase fewer silicone pouches. The product can be safely washed after usage and the silicone top tube can be easily changed every 24-48 hours.

### OBJECTIVES OF THE INVENTION

The main objective of the present invention is to promote maternal-infant bonding through skin-to-skin contact by allowing the baby to breastfeed at the breast hands-free, giving the mother more ability to stroke and hold the baby.

The other object is to promote milk supply by stimulating the breast through sustained sucking in such a way that when the baby sucks on the breast, it sends a signal to the brain to release the hormone prolactin, which stimulates milk production through oxytocin. The supplemental nursing system helps to ensure that the baby is sucking on the breast for a prolonged period, which can help to increase milk supply.

The other objective is to support non-lactating, adoptive, surrogate, same-sex, non-binary, transgender, low-income, low-literacy, poor-health parents to create a breastfeeding/chest feeding experience wherein the leakproof supplemental nursing system can be used by any parent who wants to breastfeed their baby, regardless of their gender, LGBTQ identification, socioeconomic status, or ability to produce milk, and can help to create a more inclusive breastfeeding experience where everyone can be successful.

The other objective is to support the baby's oral development of sucking skills to create a vacuum in which the supplemental nursing system helps to train the baby to suck properly by creating a vacuum at the breast which is important for the baby's oral development and can help to prevent problems such as tongue-tie and lip-tie.

The other objective is to provide milk supplementation without the use of bottles and teats as the supplemental nursing system does not use bottles or teats, which can help to prevent nipple confusion which is important for babies who are still learning how to breastfeed.

The following summary is an explanation of some of the general inventive steps for the system, method, architecture, and container in the description. This summary is not an extensive overview of the invention and does not intend to limit the scope beyond what is described and claimed as a summary.

### SUMMARY OF THE INVENTION

The present invention describes a Supplemental nursing system. A leakproof Supplemental nursing system, having a silicone squeeze pouch with an airtight seal and a connecting silicone tube with a flow controller.

The preceding summary is intended to serve as an introduction to some embodiments of the invention. It is not meant to be an overview of all inventive subject matter disclosed in this specification. The detailed description that follows and the drawings that are referred to in the detailed description will further describe the embodiments described in the summary as well as other embodiments. Accordingly, to understand all the embodiments described in this document, a full review of the summary, detailed description, and drawings is needed.

### BRIEF DESCRIPTION OF THE DRAWINGS

The novel features believed to be characteristic of the illustrative embodiments are outlined in the appended claims. The illustrative embodiments, however, as well as a preferred mode of use, further objectives, and descriptions thereof, will best be understood by reference to the following detailed description of one or more illustrative embodiments of the present disclosure when read in conjunction with the accompanying drawings.

The accompanying picture represents the prototype representation of a waist belt with suspenders:

FIG. 1 is a representation of a leakproof Supplemental nursing system as described in the present invention.

FIG. 2 is a representation of a portion of the leakproof Supplemental nursing system.

FIG. 3 is a representation of a portion of the leakproof Supplemental nursing system.

FIG. 4 is a representation of a portion of the leakproof Supplemental nursing system.

FIG. 5 is a representation of a portion of the leakproof Supplemental nursing system.

FIG. 6 is a representation of a portion of the leakproof Supplemental nursing system.

FIG. 7 is a representation of a portion of the leakproof Supplemental nursing system.

### DESCRIPTION OF THE INVENTION

In the following detailed description of the invention, numerous details, examples, and embodiments of the invention are described. However, it will be apparent to those knowledgeable in the field that the invention is not limited to the embodiments set forth and that the invention can be adapted for a wider range of applications.

The present invention presents a Supplemental nursing system. It describes a leakproof Supplemental nursing system, comprising a silicone squeeze pouch with an airtight seal and a connecting silicone tube with a flow controller.

Mothers trying to breastfeed their babies often experience challenges that interfere with their confidence, such as a low milk supply or a baby not latching well.

### Invention

The invention introduces a leak-proof supplemental nursing system that would be more effective and easier to use than the other supplemental nursing systems. The improved supplemental nursing system would have the following features:

A leak-proof BPA-free lid 2 instead of the artificial nipple that is normally used, wherein the lid 2 is configured to be received within a cap 5 Including an attached airtight tube 3 inserted that can be changed out every 24-48 hours for safety.

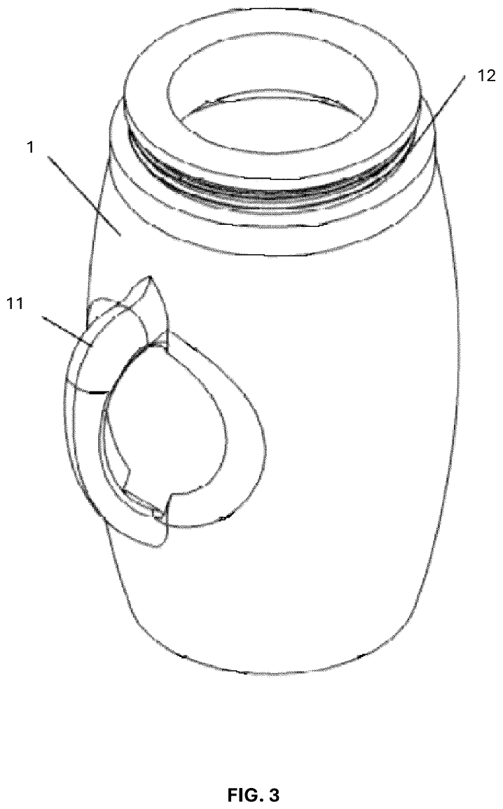

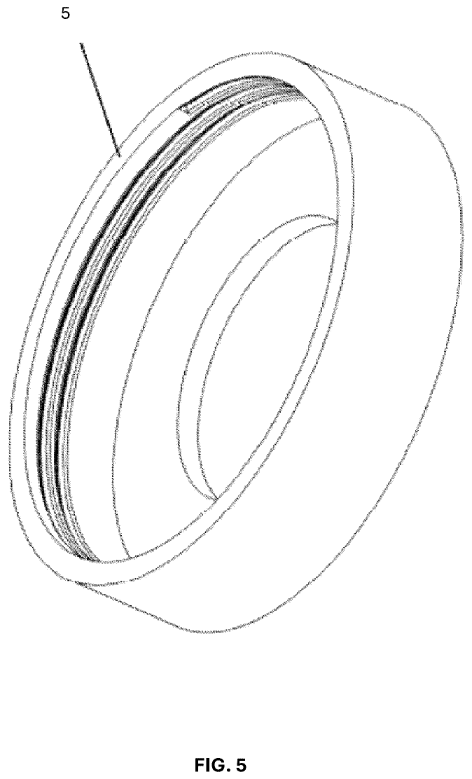

A silicone BPA-free milk storage pouch 1 is used for easy suction and cleaning. This pouch 1 includes a handle 11 for hanging on taller surfaces for easy gravitation. The pouch 1 further includes threads 12 (FIG. 3) configured to threadably engage a corresponding internally-threaded profile provided on the cap 5 (as shown in FIG. 5).

A thin tube 3 that is easy to clean and has a flow control attachment 4 that can adjust the supplemental milk from a drip flow to a medium or a faster flow. The tube 3 may be operatively coupled to the lid 2 via an orifice 7.

A plastic mobile arm 6 that can attach to the handle 11 from a silicone pouch 1 to keep the mother's hands free for the ability to reposition the baby if needed.

In addition to these improvements, the supplemental nursing system also has several other features that make it unique, such as:

A silicone pouch 1 with ounces labeled that enables you to easily keep track of how much baby is drinking.

An adjustable flow rate that allows mothers to control how much milk is delivered to their babies from drip speed to constant flow.

A leak-proof design that prevents milk from leaking out of the bottle.

A handle 11 on the silicone pouch 1 enables you to hang and feed anywhere.

An optional easy assemble versatile plastic handle that can be used to hang the silicone pouch and changed out to use as a nursery mobile.

The supplemental nursing system invention is a significant improvement over existing technology. It is more adjustable, versatile, and affordable, and has several unique features that make it a valuable tool for mothers and babies.

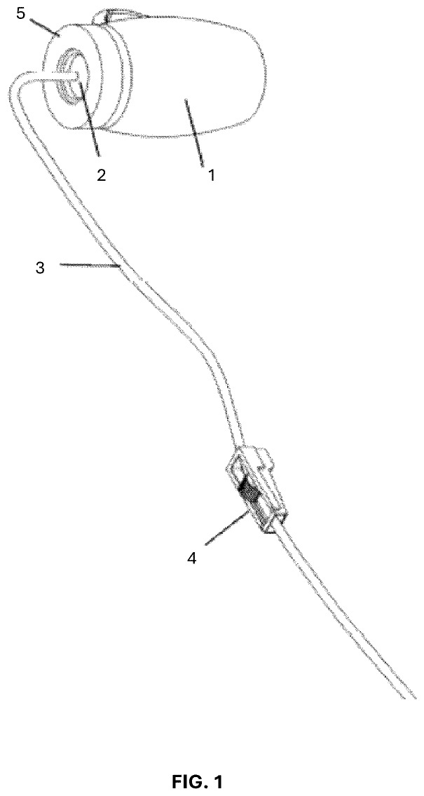

In FIG. 1, the invention describes a Supplemental nursing system with a silicone pouch as described in the present invention.

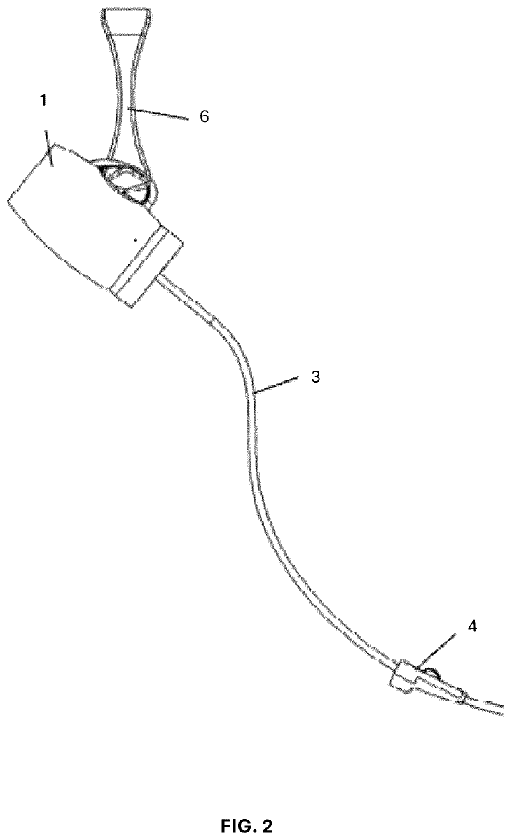

In FIG. 2, the invention describes a leakproof Supplemental nursing system.

In FIG. 3, the invention describes a leakproof Supplemental nursing system.

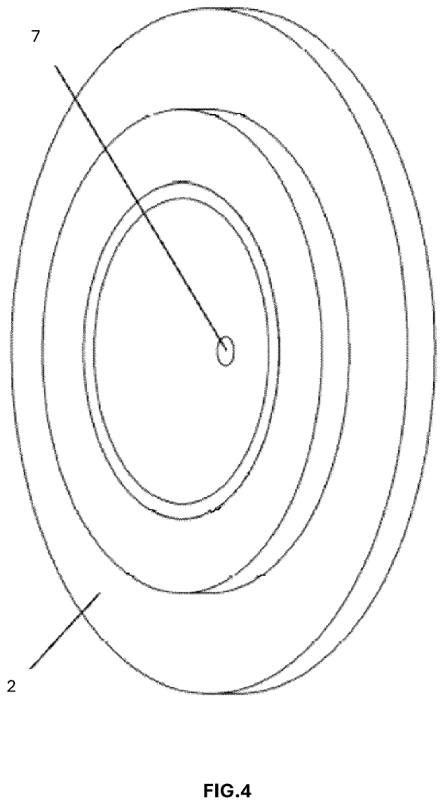

In FIG. 4, the invention describes a leakproof Supplemental nursing system.

In FIG. 5, the invention describes a leakproof Supplemental nursing system.

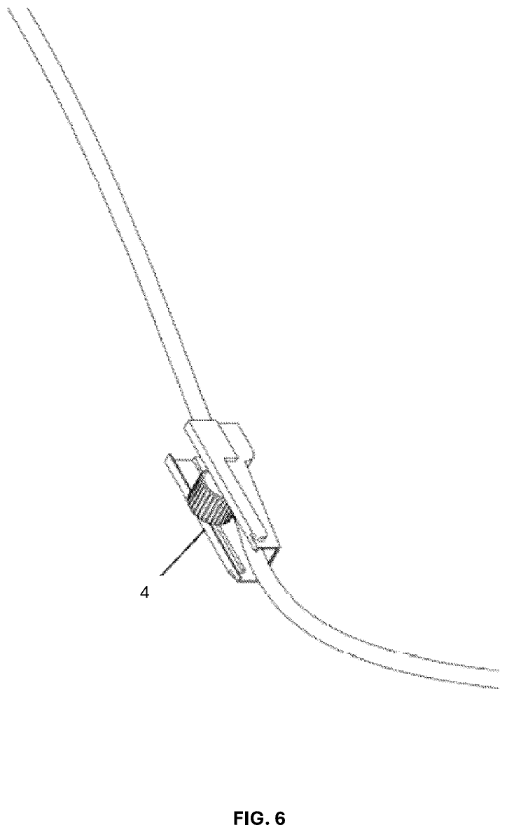

In FIG. 6, the invention describes a leakproof Supplemental nursing system.

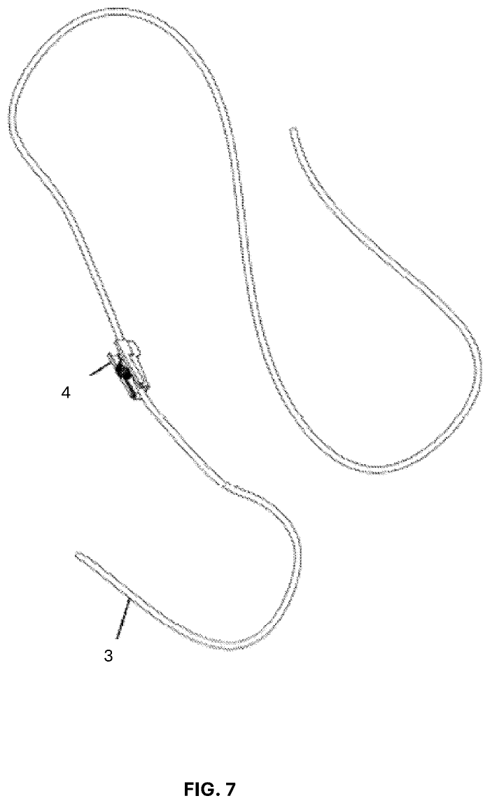

In FIG. 7, the invention describes a leakproof Supplemental nursing system.

Several individual and business demographics would use an supplemental nursing system invention. These include:

### Individuals:

Mothers who are struggling to breastfeed or who have recently returned to work.

Mothers who have low milk supply.

Mothers who can't afford to fully supplemental feed.

Mothers who are unable to pay for a lactation consultant.

Mothers who have babies who are not latching or sucking effectively.

Mothers who want to promote bonding with their babies.

Mothers without family support.

Mothers who want to increase their milk production.

Mothers who want to help their babies gain weight.

### Businesses:

Pediatricians.

Pediatric Dietitians in low-income areas.

Online retailers.

Non-profit breastfeeding organizations.

### WIC

The leak-proof supplemental nursing system invention would be a valuable tool for a wide range of individuals and businesses. It would help to make breastfeeding more successful for mothers and babies, and it would be a valuable addition to the lactation market.

Here are some specific demographics for individuals and businesses that would use a supplemental nursing system invention:

### Individuals

Age: Mothers of all ages would benefit from using a supplemental nursing system invention.

Income: The Leakproof supplemental nursing system invention is low-cost and reusable, so everyone can afford it.

Education: Mothers with all levels of education would benefit from using a hassle-free Leakproof supplemental nursing system invention.

Location: Mothers from all over the world would benefit from using a supplemental nursing system invention, especially in countries where success rates are the lowest.

### Businesses

Type of business: WIC, pediatricians, non-profit breastfeeding organizations, and online retailers would all benefit from selling or distributing Leakproof supplemental nursing system inventions.

Size of business: Leakproof supplemental nursing system invention could be sold or distributed by businesses of all sizes.

Location: The Leakproof supplemental nursing system invention could be sold or distributed by businesses in all parts of the world.

### Benefits

The new supplemental nursing system would have many benefits over traditional supplemental nursing systems. These benefits include:

Increased milk absorption for the baby.

Increased breastfeeding success of mothers.

Easy assembly and no learning curve needed therefore increasing convenience.

Reduced stress for mothers. Low stress increases milk supply.

More comfortable: Existing supplemental nursing system systems can be uncomfortable for some mothers and babies. With a Leakproof supplemental nursing system, you can relax without having to sit up the entire time since moving can cause leakage.

More versatile: Existing supplemental nursing systems are typically designed for one specific purpose, which is increasing milk supply or helping babies who are not latching. My Leakproof supplemental nursing system is designed to be more versatile, you can change out the top of the pouch to a nipple and use it for bottle feeds.

More affordable: Existing supplemental nursing system systems can be expensive. My Leakproof supplemental nursing system is designed to be more affordable, so it can be accessible to everyone.

Hassle-Free: Every supplemental nursing system in the market needs a lactation consultant to teach you how to use their product. This product is easy to understand without a learning curve or milk spills.

### CONCLUSION

I believe that the new supplemental nursing system that I have invented would be a valuable tool for mothers who want to breastfeed their babies. The leakproof supplemental nursing system would be more effective and simpler to use than traditional supplemental nursing systems, and it would offer many benefits for both mothers and babies.

While the invention has been described concerning numerous specific sensors, one of ordinary knowledge in the field will recognize that the invention can be embodied in other specific forms without departing from the spirit of the invention. Specific operations may not be performed in one continuous series of operations, and different specific operations may be performed in different embodiments. Furthermore, the processes or methods could be implemented using several sub-processes, or as part of a larger macro process. Thus, one of ordinary knowledge in the field would understand that the invention is not to be limited by the foregoing illustrative details, but rather is to be defined by the appended claims.

### INDUSTRIAL APPLICATION

The present invention applies to a Supplemental nursing system. It introduces a leakproof Supplemental nursing system, comprising a silicone squeeze pouch with an airtight seal and a connecting silicone tube with a flow controller. Type of business includes WIC, pediatricians, non-profit breastfeeding organizations, and online retailers would all benefit from selling or distributing leakproof supplemental nursing system inventions. Size of business Leakproof supplemental nursing system invention could be sold or distributed by businesses of all sizes. The location of the leak-proof supplemental nursing system invention could be sold or distributed by businesses in all parts of the world.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, uphole, downhole and the like are used in relation to the illustrative embodiments as they are depicted in the figures, the upward direction being toward the top of the corresponding figure and the downward direction being toward the bottom of the corresponding figure, the uphole direction being toward the surface of the well and the downhole direction being toward the toe of the well.

What is claimed is:

1. A leakproof supplemental nursing system, comprising:
a silicone squeeze pouch with an airtight seal; and
a connecting silicone tube with a flow controller.

2. The leakproof supplemental nursing system according to claim 1, further comprises a leakproof BPA-free lid instead of the artificial nipple that is normally used.

3. The leakproof supplemental nursing system according to claim 2, further comprises an attached airtight tube inserted that can be changed out every 24-48 hours for safety.

4. The leakproof supplemental nursing system according to claim 1, further comprising a silicone BPA-free milk storage pouch is used for easy suction and cleaning. This pouch includes a handle for hanging on taller surfaces for easy gravitation.

5. The leakproof supplemental nursing system according to claim 1, further comprises a thin tube that is easy to clean and has a flow control attachment that can adjust the supplemental milk from a drip flow to a medium or a faster flow.

6. The leakproof supplemental nursing system according to claim 1, further comprises a plastic mobile arm that can attach to the handle from a silicone pouch to keep the mother's hands-free for the ability to reposition the baby if needed.

7. A leakproof supplemental nursing system, comprising:
a silicone pouch;
an adjustable flow rate;
a leak-proof design;
a handle on the silicone pouch; and
an optional easy assemble versatile plastic handle.

8. The leakproof supplemental nursing system of claim 7, wherein the silicone pouch has ounces labeled that enables you to easily keep track of how much baby is drinking.

9. The leakproof supplemental nursing system of claim 7, wherein the adjustable flow rate is designed to allow mothers to control how much milk is delivered to their babies from drip speed to constant flow.

10. The leak-proof Supplemental Nursing System according to claim 7,
wherein the leak-proof design is crafted to prevent milk from leaking out of the bottle.

11. The leakproof supplemental nursing system of claim 7, wherein the handle on the silicone pouch enables you to hang and feed anywhere.

12. The leakproof supplemental nursing system of claim 7, wherein the optional assemble versatile plastic handle can be used to hang the silicone pouch and changed out to use as a nursery mobile.

13. The leakproof supplemental nursing system of claim 17,
a valuable tool for mothers who want to breastfeed their babies;
effective and simpler to use than traditional supplemental nursing systems and offers many benefits for both mothers and babies;
a breastfeeding product designed to assist mothers in breastfeeding their babies while also supplementing with formula or pumped breast milk;
designed to work by providing a small amount of milk to the baby through a tube that is inserted into the corner of their mouth while they are nursing on the mother's nipple; and
prepared to help support the baby to get the milk it needs while also stimulating the mother's breasts to produce more milk.
